(19) United States
(12) Patent Application Publication
(10) Pub. No.: US 2003/0032083 A1
(43) Pub. Date: Feb. 13, 2003

(54) Peptide synthesis method

(75) Inventors: Mark J. Burk, San Diego, CA (US); Grace DeSantis, San Diego, CA (US)

Correspondence Address: HALE AND DORR, LLP, 60 STATE STREET, BOSTON, MA, 02109

(73) Assignee: Diversa Corporation, San Diego, CA

(21) Appl. No.: 10/154,184

(22) Filed: May 23, 2002

Related U.S. Application Data
(60) Provisional application No. 60/292,852, filed on May 23, 2001.

(51) Int. Cl. C12P 21/06

(52) U.S. Cl. 435/68.1; 530/332

(57) ABSTRACT

Disclosed are methods for sequential peptide synthesis which reduce the potential side reactions and by-products formed in the process. These methods utilize enzymes for the coupling and deprotection steps.

Also disclosed are methods for identifying a deprotection enzyme useful in such peptide synthesis methods.

### CROSS REFERENCE TO RELATED APPLICATIONS

This application claims priority to U.S. provisional application serial no. 60/292,852, filed May 23, 2001, the entire contents of which are hereby incorporated by reference.

### BACKGROUND OF THE INVENTION

1. Field of the Invention

This invention relates the field of peptide synthesis. More particularly, this invention relates to methods of synthesizing peptides.

2. Background of the Invention

Peptides can be potent mediators of biological activity in systems as diverse as sexual maturation and reproduction, blood pressure regulation, glucose metabolism, central nervous system regulation, thermal control, enzyme inhibition and analgesia (Ferraiolo et al. Pharm Res. 4:151-156, 1985). Thus, the peptide therapeutic market is rapidly growing. The manufacture of the active pharmaceutical ingredient (API) for peptide therapeutics is an important component of the market. Currently, peptide API manufacturing cost contributes a significant portion of the cost of the final drug (Latham, P. W., Nature Biotechnology 17:755-757, 1999).

Peptides may be synthesized by several different methods, including ribosomally-directed fermentation methods, as well as non-ribosomal strategies and chemical synthesis methods. Peptides containing the 20 natural amino acids and those greater than about 30 residues can be prepared via recombinant expression systems that utilize the ribosomally directed peptide synthesis machinery of a host organism, e.g., E. coli. However, development of efficient peptide overexpression systems often requires tedious and protracted optimization studies, and the number of peptides that may be produced efficaciously by these recombinant techniques actually is quite limited. For example, very small peptides (less than 10 residues) are difficult or impossible to produce through ribosomal pathways.

Smaller peptides (less than 30 residues) and peptides which contain unnatural or non-proteninogenic amino acids or modified amino acid side chains are also difficult to prepare ribosomally. Thus, these peptides are often prepared through a more general solution-phase chemical synthesis of peptides (e.g., using N-Boc protection and the activated ester route). Protocols for sequence solution-phase chemical synthesis of peptides have been described (see, e.g., Andersson et al., Biopolymers 55:227-250, 2000). One current method used for generating peptides is solution-phase chemical synthesis, which employs a N-tert-butoxy (N-Boc) protected amino acid and a C-protected amino acid (Andersson et al., Biopolymers 55: 227-250, 2000). An alternative solution-phase method for chemically-catalyzed peptide synthesis employs pre-activated esters as the carboxyl component for coupling (Andersson et al., Biopolymers 55: 227-250, 2000).

In addition, enzyme-mediated solid-phase peptide synthesis has also been employed. Solid-phase peptide synthesis (SPPS) uses insoluble resin supports, and has simplified and accelerated peptide synthesis and facilitated purification (Merrifield, R.B., J. Am. Chem. Soc. 85: 2149-2154, 1963). Since the growing peptide is anchored on an insoluble resin, unreacted soluble reagents can be removed by simple filtration or washing without manipulative losses. Solid phase peptide synthesis can be performed using automation.

However, the current protocols for sequential solution-phase chemical synthesis of peptides suffer from numerous disadvantages such as: (1) requirement at each coupling step for expensive carboxyl-activating groups or stoichiometric quantities of coupling reagents (e.g., DCC and HOBt); (2) racemization of carboxyl donor amino acids during coupling; (3) need for expensive side-chain, and N- and C-protecting groups (e.g., Cbz, Boc); (4) requirement for harsh deprotection procedures (e.g., TFA, HCl, and hydrogen pressure) to be carried out at each step; (5) serious side reactions or peptide degradation during coupling and deprotection procedures; and (6) extensive waste produced at each step of the synthesis (e.g., urea from DCC, aqueous HC1 or trifluoroacetic acid and anisole from deprotection, and by-products from incomplete coupling).

Thus, there remains a need to develop a convenient and general peptide synthesis method that may be utilized on commercial scales (e.g., to produce more than 1 kg of peptide) and which obviates the litany of problems listed above. Such a method potentially could replace all chemical procedures currently being employed for commercial peptide manufacture.

### SUMMARY OF THE INVENTION

The invention relates to a convenient and general peptide synthesis method that may be utilized on commercial scales (e.g., to produce more than 1 kg of peptide) and which obviates the numerous problems of previous methods. The invention provides a method for the synthesis of peptides of 2 to 40 residues or 4 to 30 residues, ranges which are of particular clinical importance. The method of the invention also permits the preparation of peptides containing unusual amino acid residues.

This method uses enzymes for both coupling and deprotection of the amino acids that form the peptide. The method of the invention is useful for the synthesis of peptides with minimal waste and minimal side reactions.

Accordingly, in one aspect, the invention provides a method for synthesizing a peptide, comprising: (a) ligating a protected amino acid carboxyl component to an amino acid nucleophilic component with a peptide-coupling enzyme active at a first temperature to produce a first peptide; deprotecting the first peptide with a deprotection enzyme active at a second temperature; and ligating a protected amino acid carboxyl component to the deprotected first peptide with the peptide-coupling enzyme active at the first temperature to produce a second peptide. In certain embodiments, steps (b) and (c) are repeated to obtain a peptide of a desired length. In some embodiments, the desired length of the resulting peptide is from about two to about thirty amino acids in length. In some embodiments, the desired length of the resulting peptide is from about three to about thirty amino acids in length.

In certain embodiments of the invention, the carboxyl component is a N-acyl protected amino acid. In some embodiments, the acyl is acetyl, formyl, benzoyl, or carbamoyl.

In particular embodiments, the protected amino acid carboxyl component consists of the formula,

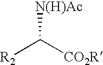

In some embodiments, the R′ group of the protected amino acid carboxyl component is hydrogen or alkyl.

In certain embodiments, the nucleophilic component is a C-terminal protected amino acid. In some embodiments, the amino acid nucleophilic component consists of the formula,

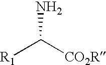

In certain embodiments, the R′ group of the amino acid nucleophilic component is t-butyl.

In particular embodiments, the peptide-coupling enzyme does not have protease activity. In some embodiments, the peptide-coupling enzyme is a peptidase or a ligase. In certain embodiments, the peptide-coupling enzyme is a protease, a lipase, a carboxypeptidase, esterase, or an amidase.

In some embodiments, the deprotection enzyme is a deacetylase. In certain embodiments, the deprotection enzyme is an aminoacylase, an amidase, a carbamate, or a carboxypeptidase.

In some embodiments, the peptide synthesis method of the invention is performed in a single reaction vessel.

In certain embodiments, the peptide-coupling enzyme and the deprotection enzyme are not active simultaneously. In particular embodiments, the first temperature is higher than the second temperature. In other embodiments, the second temperature is higher than the first temperature.

In some embodiments, the peptide-coupling enzyme has an optimum activity at the first temperature. In certain embodiment, the peptide-coupling enzyme is inactive at the second temperature, while in some embodiments, the peptide-coupling enzyme is not irreversibly inactivated at the second temperature. In some embodiments, the deprotection enzyme has an optimum activity at the second temperature. In certain embodiment, the deprotection enzyme is inactive at the first temperature, while in some embodiments, the deprotection enzyme is not irreversibly inactivated at the first temperature.

In certain embodiments, the peptide-coupling enzyme, the deprotection enzyme, or both the peptide-coupling enzyme and the deprotection enzyme are from a thermophilic organism.

In another aspect, the invention provides a method for identifying a deprotection enzyme. The method according to this aspect includes culturing a plurality of host cells on a substrate comprising an acetylated peptide as a sole carbon source, wherein each host cell comprises a nucleic acid molecule suspected of encoding a deprotection enzyme, and wherein a host cell that grows on the substrate is identified as a host cell that expresses a deprotection enzyme.

### BRIEF DESCRIPTION OF THE DRAWINGS

FIG. 1 is a schematic representation showing a non-limiting embodiment of the peptide synthesis method of the invention. Sequential coupling and deprotection steps are effected by thermal cycling of a reactor. Coupling is conducted at 50° C., and the reactor is cooled to 20° C. in order to unmask (i.e., deprotect) the next reactive group.

FIG. 2 is a schematic representation showing a non-limiting thermal-cycled bioreactor configuration for use in one embodiment of the peptide synthesis method of the invention. The reactor is equipped with a pH-probe and biurette in order to regulate the pH of the reaction solution, as necessary.

FIG. 3 is a schematic representation showing a non-limiting two column embodiment of the peptide synthesis method of the-invention. In-accordance with this embodiment, the enzymes immobilized and the reaction solutions are cycled from one column to another.

FIG. 4 is a schematic representation showing the active site nomenclature of the hydrolases of the invention. The substrate residues are denoted P₁, P₂ etc. and the active site pockets to which they bind are denoted S₁, S₂ etc. The residues on the carbonyl side of the scissile bond are denoted P₁′, P₂′ etc.

FIG. 5 is a schematic representation showing the resolution of N-acetyl-methionine by an A. oryzae aminoacylase, a non-limiting deprotection enzyme of the invention.

FIG. 6 is a is a schematic representation showing a non-limiting example of the one enzyme embodiment of the peptide synthesis method of the invention.

FIG. 7 is a schematic representation showing the acyl-transfer reaction catalyzed by a non-limiting peptide ligase of the invention. Starting with the typical serine hydrolase template for which catalysis proceeds by way of an acyl enzyme intermediate, a number of reactions may follow formation of the active acyl enzyme. A peptide ligase with very high aminolysis to hydrolysis activity and with very high acyl-transferase to proteolytic activity is useful in the invention.

FIG. 8A is a schematic representation showing the application of ninhydrin for detection of amino amide substrate remaining, as an assessment of peptide ligation. Note that this method may not be applicable to detection of amino amide substrates since for amino acids formation of Ruhemann's purple is dependent upon decarboxylation.

FIG. 8B is a schematic representation showing the application of ortho-phthalaldehyde (OPA) for detection of amino amide substrate remaining, as an assessment of peptide ligation.

FIG. 9 is a diagrammatic representation showing the growth selection for the discovery of deacetylases, a non-limiting method for identifying a deprotection enzyme of the invention.

FIG. 10 is a schematic representation showing the non-limiting thermally-cycled dual enzyme peptide synthesis embodiment of the method of the invention.

### DETAILED DESCRIPTION

The patent and scientific literature referred to herein establishes knowledge that is available to those with skill in the art. The issued patents, published patent applications, and other references, including GenBank database sequences, that are cited herein are hereby incorporated by reference to the same extent as if each was specifically and individually indicated to be incorporated by reference.

The present invention presents an enzymatic method for sequential peptide synthesis, which is both a general and efficient method. Such methods of the invention provide alternatives for chemical procedures currently being employed for commercial peptide manufacture. One non-limiting improvement of the invention involves the use of enzymes for peptide synthesis.

Enzymatic procedures for peptide bond formation have been studied extensively. For example, numerous proteases such a chymotrypsin, thermolysin, papain, subtilisin, and carboxypeptidase Y have been demonstrated to be useful for the synthesis of specific peptides. Lipases, esterases, and amidases also have been used for peptide bond formation. Enzymes such as lipases, esterases, and aminoacylases also have been used to deprotect amino acids and peptides. The noted advantages of enzymatic peptide synthesis are: (1) the absence of carboxyl donor racemization; (2) mild conditions employed for coupling and deprotection steps; (3) minimal protecting groups required; (4) reduced use of organic solvents; (5) avoidance of expensive and toxic coupling reagents; (6) biocatalyst recycle and reuse (e.g., through immobilization); and (7) reduced waste production. However, enzymes currently being used for peptide synthesis also exhibit several critical deficiencies. These include severely restricted amino acid substrate tolerance (e.g., only specific natural L-amino acids are coupled efficiently), inherent protease activity leading to peptide bond hydrolysis, low volume productivities, and enzyme instability in range of solvents.

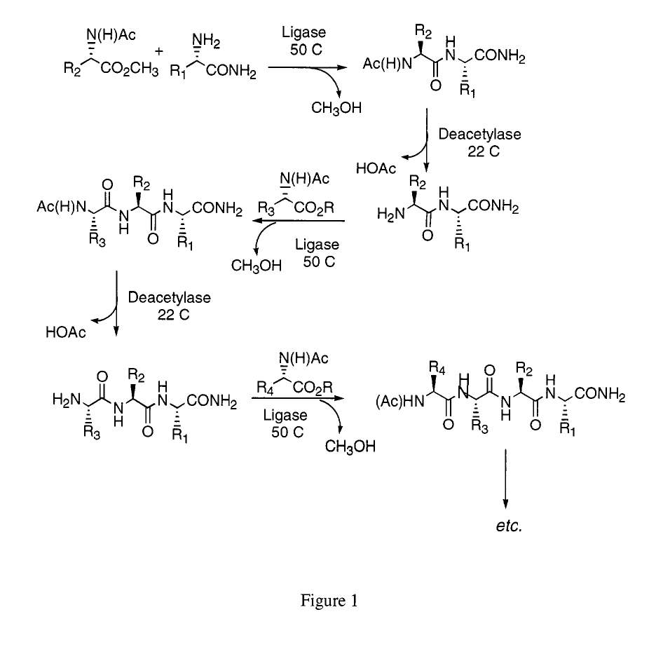

A peptide made in accordance with the method of the invention is constructed from C- to N-terminus and the first N-terminal residue is differentially protected. Where the peptide is made in a single reaction vessel, the reactor is heated to a first temperature prior to the addition of each subsequent amino acid. The ligation reaction is catalyzed by a peptide-coupling enzyme and is conducted at the first temperature. After the ligation reaction is complete, the reactor is cooled to a second temperature, allowing deprotection of the N-terminal amino acid by deprotection enzyme (e.g., a deacetylase) to proceed. Once the deprotection is complete, the reactor is then again heated to the first temperature and the next amino acid added. The cycle is repeated until the desired chain length peptide is synthesized (see FIG. 1).

Note that the temperatures at which the peptide-coupling enzyme and the deprotection enzyme are active can be reversed to those listed in FIG. 1. What is relevant in this embodiment of the invention is that thermocycling is used to control the synthesis of the growing peptide. Thermal enzymatic synthesis of a peptide in accordance with this embodiment of the invention (as compared to synthesis at room temperature) is useful because, among other things, it overcomes folding problems which can inhibit synthesis (e.g., the peptide may fold back on itself and block synthesis). Another non-limiting advantage of thermocycling peptide synthesis is that it overcomes peptide solubility problems which limit the effectiveness of current peptide synthesis methods (e.g., the synthesis steps cannot be performed if the peptide is insoluble).

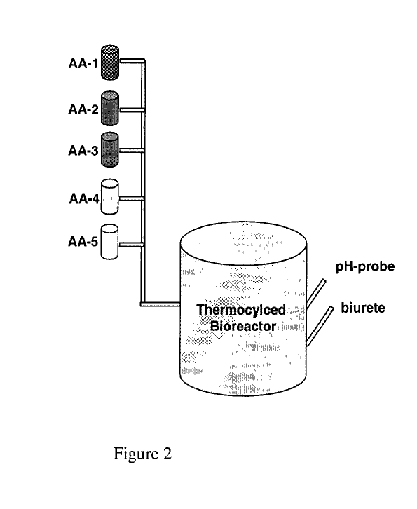

Thus, the method of the invention provides for sequential peptide synthesis, where enzymes are used in both the coupling and deprotection steps. The use of enzymes for both the coupling and deprotection steps reduces the potential side reactions and by-products formed in the process. In certain embodiments, the synthesis (e.g., including both peptide-coupling and deprotection steps) occurs in a single reaction vessel. A non-limiting reactor for use in the invention is generically depicted in FIG. 2.

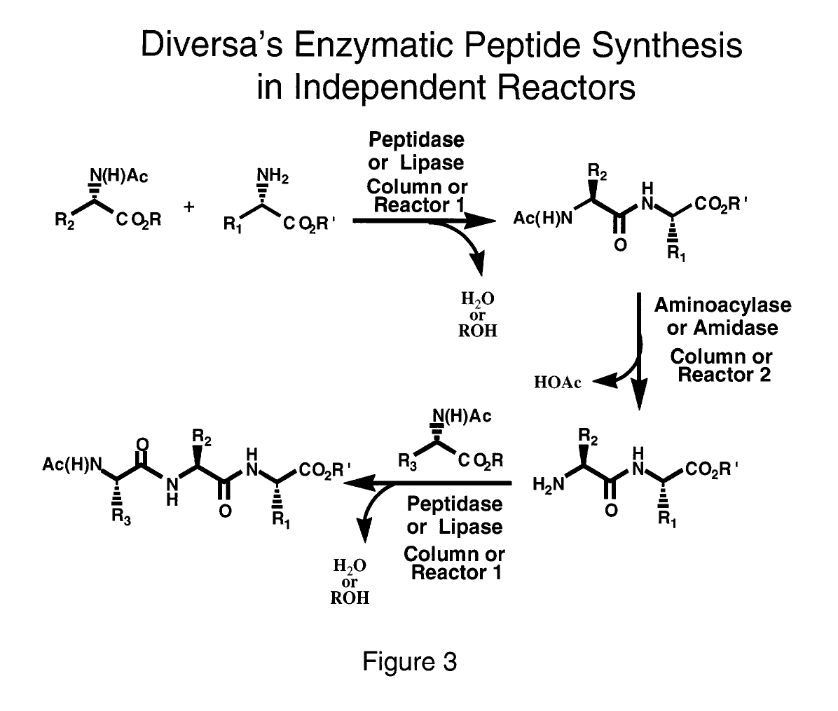

In another embodiment of the sequential peptide synthesis method of the invention, control of the peptide-coupling step and the deprotection step can be through the use of a two column (or two reactor) system (e.g., ligating on one column and deprotecting on a second column). In this embodiment, the enzymes are localized in their respective columns (e.g., covalently bonded to the side of a column), and the growing peptide is transferred back and forth from the peptide-coupling column to the deprotection column. A non-limiting example of this embodiment is generically depicted in FIG. 3. In accordance with this embodiment, both enzymes can operate at same or different temperatures. Moreover, the same reactor can contain both D- and L-specific enzymes.

The methods of the present application are equally applicable to both solution phase and solid phase peptide synthesis.

Accordingly, in one aspect, the invention provides a method for synthesizing a peptide, comprising: (a) ligating a protected amino acid carboxyl component to an amino acid nucleophilic component with a peptide-coupling enzyme active at a first temperature to produce a first peptide; deprotecting the first peptide with a deprotection enzyme active at a second temperature; and ligating a protected amino acid carboxyl component to the deprotected first peptide with the peptide-coupling enzyme active at the first temperature to produce a second peptide.

In certain embodiments, steps (b) and (c) are repeated to obtain a peptide of a desired length. Because the method of the invention adds a protected amino carboxyl component to the growing peptide, only one amino acid will be added to the growing peptide with each cycle. Thus, the sequence of the resulting peptide of the invention is readily controllable.

It will be understood that the method of the invention allows the generation of any peptide larger than two amino acids in length. Note that to obtain a three amino acid long peptide (i.e., a tripeptide), steps (b) and (c) are simply not repeated. To obtain longer peptides (e.g., a peptide consisting of four amino acids), steps (b) and (c) are repeated x number of times to obtain a peptide of n amino acids,-according to the following formula:

n=3+x

Thus, to obtain a ten amino acid long protected peptide (i.e., n=10), steps (b) and (c) are repeated seven times.

Of course, the ordinarily skilled artisan will realize that should a deprotected peptide be desired as the final product, the above method is simply extended for an extra step (b) without the following step (c). Thus, in the above example for a ten amino acid long peptide, the steps (b) and (c) are repeated seven times, and then an addition step (b) is performed to deprotect the ten amino acid long peptide.

In accordance with the invention, a peptide of any length greater than or equal to two amino acids is readily obtainable. Similarly, a peptide of any length greater than or equal to three amino acids is readily obtainable according to the invention. In certain embodiments, the desired length of the resulting peptide is between about 2 to about 30 amino acids, between about 2 to about 25 amino acids, between about 2 and about 20 amino acids, between about 2 to about 15 amino acids, or between about 2 and about 10 amino acids in length. In certain embodiments, the desired length of the resulting peptide is between about 3 to about 30 amino acids, between about 3 to about 25 amino acids, between about 3 and about 20 amino acids, between about 3 to about 15 amino acids, or between about 3 and about 10 amino acids in length. The term “about” is used herein to mean “approximately,” or “roughly,” or “around,” or “in the region of.” When the term “about” is used in conjunction with a numerical range, it modifies that range by extending the boundaries above and below the numerical values set forth. In general, the term “about” is used herein to modify a numerical value above and below the stated value by a variance of 20 percent. Thus, the phrase “about ten amino acids” is construed to mean that a 20% variance in value is placed on the numeral “ten”; for example, “about ten amino acids” refers to all values between 10 minus (0.2) (10) and 10 plus (0.2) (10) (i.e., “about ten amino acids” refers to between 8 and 12 amino acids).

In certain embodiments, the method of the invention is performed in a single vessel.

As used herein, an “amino acid” or “amino acid residue” refers to naturally occurring or synthetic amino acid molecules, as well as amino acid analogs. The amino acid molecules may be α-amino acids or β-amino acids, γ-amino acids, δ-amino acids, and may be D(R)- or L(S)-amino acid, or may be an amino acid analog.

The term, an “amino acid carboxyl-component” or simply “carboxyl component” (also simply called an “amino acid donor”) refers to an amino acid, α-hydroxy acid or β-hydroxy acid, of which the carboxyl group is involved in the coupling reaction. An “amino acid carboxyl component” has the following generic formula:

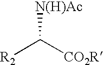

In some embodiments, an amino acid carboxyl component has the following generic formula:

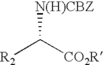

The OR′ group of the carboxyl of the amino acid carboxyl component is removable, to form a positively charged carboxyl group. The carboxyl group retains the carbon-oxygen double bond of the carboxyl group in forming the new peptide. In certain embodiments of the invention, the carboxyl component is a N-acyl protected amino acid (see, e.g., FIG. 1), the acyl of the carboxyl component being, for example, acetyl, formyl, benzoyl, or carbamoyl. The amino acid of the carboxyl component has a R′ group of hydrogen or an alkyl group in some embodiments of the invention.

As used herein, an “amino acid nucleophilic component” or simply “nucleophilic component”(also simply called an “amino acid acceptor”) refers to an amino acid which is an electron donor in a reaction. In the present invention, the nucleophilic component is an amino acid of which the amino group is involved in the coupling reaction. An “amino acid nucleophilic component” has the following generic formula:

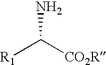

The R″ group attached to the carboxyl group of the amino acid of the nucleophilic component is t-Butyl in some embodiments of the invention.

In some embodiments, the nucleophilic component has the generic formula:

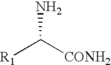

One of the hydrogens of NH₂ group of the nucleophilic component is removed to form a negatively charged ion which accepts the positively charged carboxyl group of the amino acid donor to form a peptide bond. In some embodiments, the nucleophilic component is a C-terminally protected amino acid. The C-terminus of the amino acid nucleophilic component may be immobilized on a solid support (e.g., an insoluble resin).

The method of the invention involves the combination (i.e., coupling or ligating) of a carboxyl component with a nucleophilic component, catalyzed by a “peptide-coupling” enzyme, to form a peptide bond. The resulting dipeptide is deprotected or deaceylated by a second “deprotection”) enzyme, resulting in an amino peptide. The amino peptide is then ready for addition of another carboxyl amino acid component, in the presence of the peptide-coupling enzyme. The resulting peptide is tripeptide. This process is repeated to achieve a polypeptide of desired length.

As used herein, a “peptide-coupling” or “coupling” or “ligating” refers to the joining of the amino acid carboxyl component and the amino acid nucleophilic component to form a peptide bond.

The term, “peptide-coupling enzyme” as used herein, means any enzyme capable of ligating an amino acid carboxyl component to an amino acid nucleophilic component to form a peptide bond. In certain embodiments of the invention, the peptide-coupling enzyme only catalyzes peptide-coupling. For example, the peptide-coupling enzyme does not have any protease activity. If it does appear that protease activity is present, organic solvents may be used, such as methanol or ethanol, which may reduce proteolysis. In certain embodiments, the peptide-coupling enzyme is a peptidase or a lipase. The peptide-coupling enzyme may also be a carboxypeptidase, an amidase, or a protease, such as a protease that does not have protease activity in a method according to the invention. One non-limiting protease useful as a peptide-coupling enzyme according-to the invention is the Subtilisin Carlsberg protease. Other non-limiting proteases are those listed on page 842 in the Sigma Chemical Co. 2000-2001 catalog (Sigma Chemical Co., St. Louis, Mo.). For example, a protease used as a peptide-coupling enzyme in accordance with the invention may have ligase activity and not protease activity. Similarly, another non-limiting peptide-coupling enzyme is an esterase, such as the porcine liver esterase or other esterases listed on pages 396-397 in the Sigma Chemical Co. 2000-2001 catalog.

In a non-limiting embodiment of the invention, it is useful that the peptide-coupling enzyme accepts at least one of an α-amino acid, a β-amino acid, a γ-amino acid, a δ-amino acid, a D(R)-amino acid, a L(S)-amino acid, and an amino acid analog as a substrate.

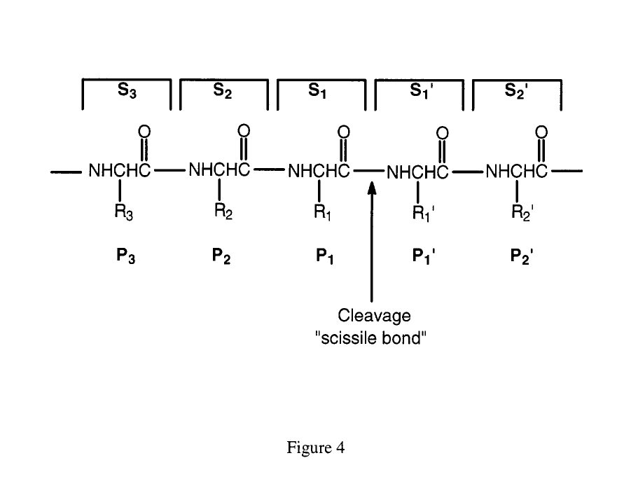

In non-limiting embodiments, a peptide-coupling enzyme of the invention has one or more of the following characteristics: (1) very efficient peptide ligase activity; (2) high aminolysis to hydrolysis ratio; (3) absence of proteolytic activity; (4) active at 50° C.; (5) stable to numerous thermal cycles; (6) broad substrate scope with respect to amino acid coupled, P₁ (see FIG. 4); (7) Broad substrate scope with respect to nucleophilic acceptor, P₁ (see FIG. 4); (8) either or both L- and D-specific; and/or (9) stable and active in methanol or ethanol.

As used herein, “deprotection” or “decetylation” refers to the removal of a protecting group from a protected amino acid. A “protected amino acid” refers to any amino acid with an attached protecting group, preventing reaction of the protected group.

The term, “deprotection enzyme” as used herein, means any enzyme capable of removing a protecting group from a protected amino acid. Non-limiting examples of deprotection enzymes of the invention include aminoacylases, amidases, carbamates, and carboxypeptidases. Where the amino acid carboxyl component is protected with a N-CBZ group, deprotection may be accomplished either with treatment with HBr/HOAc/anisol or hydrogenation using a metal catalyst PD/c under hydrogen pressure. Where the amino acid carboxyl component is protected with a N-tert-butoxy group (N-Boc), deprotection may be accomplished in acidic conditions, such a trifuluoroacetic acid or hydrocholic acid together with a carbocation scavenger such as anisole (see, e.g., Greene, T. W. and Wuts, P. G. M., Protective Groups in Organic Synthesis, 3^{rd} Ed., 1999). It will be understood, however, that any X-carboxyl hydrolyzing enzyme is useful as a deprotection enzyme in accordance with the invention. Additional useful deprotection enzymes of the invention include, without limitation, N-carboxyl hydrolyzing enzymes, N-carbonyl cleaving-enzymes, and N-acyl cleaving enzymes. In a non-limiting embodiment of the invention, it is useful that the deprotection enzyme accepts at least one of an α-amino acid, a β-amino acid, a D(R)-amino acid, and a L(S)-amino acid as a substrate.

In non-limiting embodiments, a deprotection enzyme of the invention has one or more of the following characteristics: (1) efficient deacetylase activity; (2) absence of primary amidase activity; (3) absence of proteolytic activity; (4) inactive at 50° C.; (5) active at 20° C.; (6) numerous thermal cycles; (7) broad substrate scope with respect to amino acid; (8) either or both L- and D-specific; and/or (9) stable and active in methanol or ethanol.

Thus, the invention also provides a method for identifying a deprotection enzyme. The method according to this aspect includes culturing a plurality of host cells on a substrate comprising an acetylated peptide as a sole carbon source, wherein each host cell comprises a nucleic acid molecule suspected of encoding a deprotection enzyme. The host cell that grows on the substrate is identified as a host cell that expresses a deprotection enzyme. In one embodiment, the host cell has been transformed with a nucleic acid molecule (e.g., on an expression plasmid) suspected of encoding a deprotection enzyme.

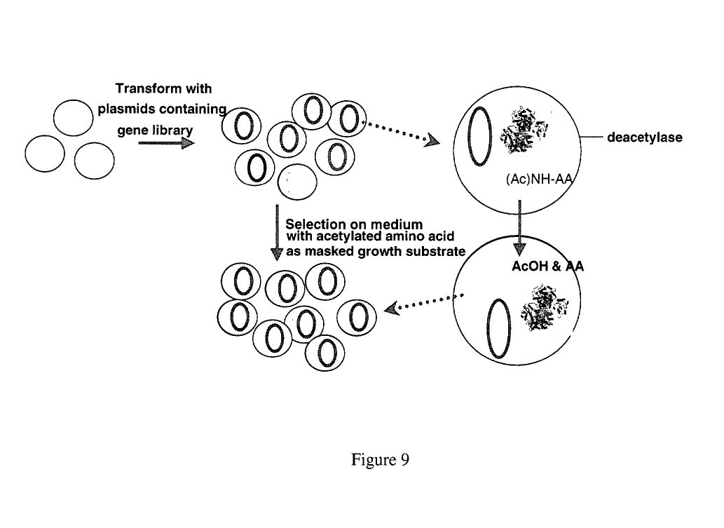

One non-limiting example of this method is described below in Example 1, and is schematically depicted in FIG. 9. Briefly, the host cells are cells transformed with a library, such as a genomic or cDNA library, from an organism having a deprotection enzyme with activity useful for the peptide synthesis method of the invention (e.g., active at 20° C., inactive at 50° C., and/or thermally tolerant, such that it is not irreversibly inactivated at 50° C.). The transformed host cells are grown on a media containing an acetylated peptide as its sole carbon source (see Table 2 for a non-limiting list of such substrates). Since the acetylated peptide is the only carbon source in the growth media, only those host cells transformed with a nucleic acid molecule encoding a deprotection enzyme will be able to grow. Those host cells can then be expanded, and the nucleic acid molecule isolated according to standard techniques (e.g., standard plasmid preparation methods described in, e.g., Ausubel et al., Current Protocols in Molecular Biology, John Wiley & Sons, New York, N.Y., 1994, and updates up to and including the year 2001).

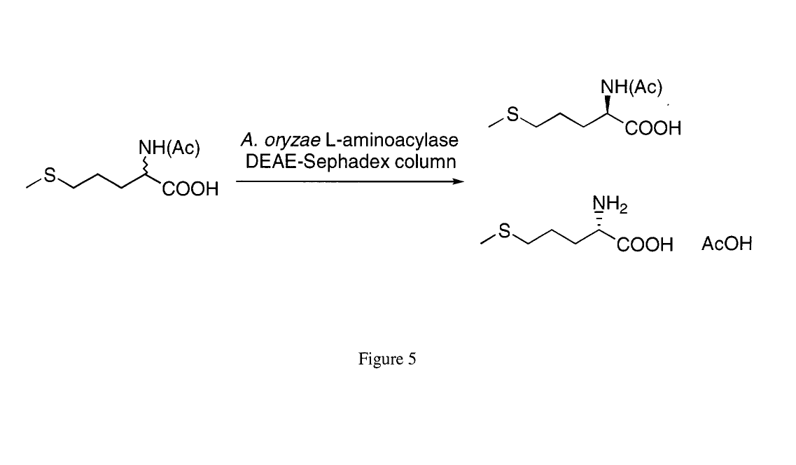

Non-limiting examples of a type of deprotection enzyme of the invention are aminoacylases, which catalyze the hydrolysis of N-acetyl amino acid derivatives. Deprotection (i.e., deacetylation) has been used successfully to effect the kinetic resolution of N-acetyl amino acids both on the laboratory scale as well as on an industrial scale using column reactors. For example, as shown in FIG. 5, the resolution of N-Ac-methionine was achieved with an A. oryzaeaminoacylase (Tosa et al., Biotechnol. Bieng. 111: 603, 1967). Aminoacylases reported in the literature thus far show a preference for acetyl, chloroacetyl or propionyl acyl groups. Thus little, if any, amidase activity is expected. Amino acylases with both L- and D-N-acyl-amino acid substrate specificities have been reported. For example, an L-N-acyl-amino acid specific aminoacylase from Asperigillus oryzae (Tosa et al., Enzymologia 31: 214, 1966) and one from Asperigillus mellus (Nettekoven et al., Tetrahedron Lett. 36: 1425, 1995) have been reported. As well, a D-N-acyl-amino acid specific aminocylase from Alcaligenes faecalis (Tsai et al., Enzyme Microb. Technol. 14: 384, 1992) has been reported and utilized in a preparative scale biotransformation. Recently, an aminoacylase was isolated from the hyperthermophilic archaeon Pyrococcus furiosus (Chen et al., Biorg. Med. Chem. 2(1):1-5, 1994) suggesting that this is a wide-spread activity. The D-aminoacylase from Alcaligenes faecalis was shown to effect the deacetylation both of a protected amino acid, N-Ac-Met-OMe and also of a dipeptide, N-Ac-Met-Gly.

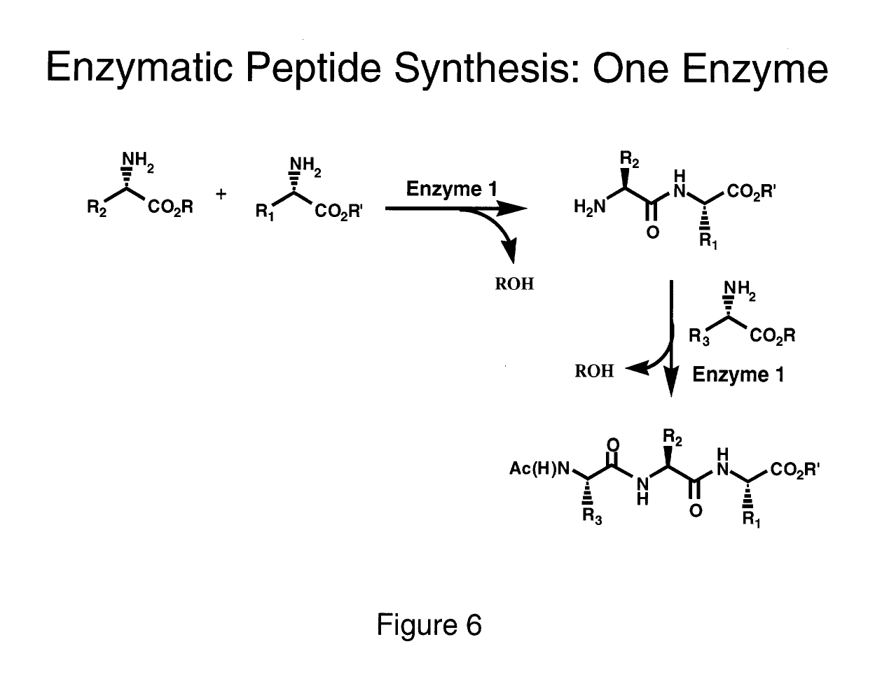

In a non-limiting embodiment of the invention, the peptide synthesis method is performed by one enzyme. In this embodiment, a single enzyme is employed which is selective for one ester leaving group, and is able to distinguish ester and amide bonds. Thus, in this embodiment, no amino protection is required, and synthesis starts at the dipeptide stage (see FIG. 6).

As used throughout, “protein,” “peptide,” or “polypeptide” are terms used interchangeably to refer to any polymer of two or more individual amino acids (whether or not naturally occurring) linked via a peptide bond, and occurs when the carboxyl carbon atom of the carboxylic acid group bonded to the -carbon of one amino acid (or amino acid residue) becomes covalently bound to the amino nitrogen atom of amino group bonded to the carbon of an adjacent amino acid. Proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins,” “peptides,” or “polypeptides”.

In some embodiments of the invention, a peptide-coupling enzyme and a deprotection enzyme of the invention are not active simultaneously. In accordance with the invention, the activity of each of the peptide-coupling enzyme and the deprotection enzyme is controllable, so that operation of each step of the method of the invention occurs independently (e.g., step (a) occurs independently of step (b)). Without the ability to control each step of the process, the protection group of the amino acids may be removed before or during coupling. Such an occurrence may compromise the fidelity of the process, leading to mixtures of peptide products.

One way to control operation of the peptide-coupling enzyme and the deprotection enzyme of the invention, and to ensure that they do not operate simultaneously (i.e., are not active simultaneously), is to regulate the operation of the each of the enzymes by temperature.

As used throughout, by an enzyme (e.g., a protein coupling enzyme or a deprotection enzyme) being “active” at a particular temperature, is meant that the enzyme is able to catalyze its reaction (i.e., combine with its substrate and chemically convert that substrate into the product) at the indicated temperature. For example, a peptide-coupling enzyme of the invention may be active at 50° C. In some embodiments, the peptide-coupling enzyme has a sharp activity optimum at or near the first temperature, and the deprotection enzyme has a sharp activity optimum at or near the second temperature. By “optimum activity” is meant that the enzyme is more active at the indicated temperature than at other temperatures.

In some embodiments of the invention, the protein coupling enzyme and the deprotection enzymes are not active at the same temperatures. Note, however, that the invention includes a protein coupling enzyme that is slightly active at the second temperature (i.e., the temperature at which the deprotection enzyme is active) and a deprotection enzyme that is slightly active at the first temperature (i.e., the temperature at which the protein coupling enzyme is active). In some embodiments, the protein coupling enzyme of the invention is inactive at the second temperature and/or the deprotection enzyme of the invention is inactive at the first temperature.

In one embodiment, the first temperature is higher than the second temperature. For example, a protein coupling enzyme of the invention may be active at about 50° C. to about 105° C. while a deprotection enzyme of the invention may be active at about 16° C. to 45° C.

In a non-limiting embodiment of the invention, the peptide-coupling enzyme is active at a higher temperature than the deprotection enzyme. In this embodiment, peptide-coupling occurs at a higher temperature than deprotection. The deprotection enzyme may be inactive at lower temperatures, so that deprotection does not occur prematurely. Once the dipeptide is formed, the temperature is lowered to activate the deprotection enzyme that deprotects the dipeptide. Once deprotection has occurred, the temperature is raised again, to promote an additional peptide-coupling. This temperature control process continues until the peptide of desired length has been formulated.

In some embodiments of the invention where the first temperature is a higher temperature than the second temperature, the deprotection enzyme is inactivated at the first temperature. In this embodiment, an amount of deprotection enzyme sufficient to deprotect the amino acid carboxyl component newly added to the growing peptide is added to the reaction vessel each time the reaction temperature is cooled to the lower temperature. An “amount sufficient to deprotect an amino acid carboxyl component” is readily determinable by any ordinarily skilled scientist, and is simply an amount that results in the deprotection of the amino acid carboxyl component to the growing peptide.

In a non-limiting embodiment of the invention where the first temperature is a higher temperature than the second temperature, the deprotection enzyme, while being inactive at the first temperature, is not irreversibly inactivated at the first temperature, such that when the temperature of the reaction is changed from the first temperature to the second temperature, the deprotection enzyme becomes active. Thus, the invention obviates the need to add new deprotection enzyme at each cycle (i.e., at each step in which a newly added amino acid is deprotected). The deprotection enzyme may be thermally stable at the first (i.e., higher) temperature. For example, the deprotection enzyme that deprotects the peptide may reversibly fold back into its active position when the temperature is lowered again. Accordingly, once the temperature is lowered, the amino acid nucleophilic component newly added to the growing peptide is deprotected without requiring the addition of deprotection enzyme to the reaction vessel.

In one non-limiting embodiment of the invention, the first temperature (i.e., at which the peptide-coupling enzyme is active) is a lower temperature than the second temperature (i.e., at which the deprotection enzyme is active). In this embodiment, peptide-coupling occurs at a lower temperature than deprotection. In some embodiments, the deprotection enzyme is not active at the first temperature at which the peptide-coupling enzyme is active. Once the dipeptide is formed, the temperature is raised, to activate-the deprotection enzyme that will deprotect the dipeptide. In certain embodiments, the peptide-coupling enzyme is not active at the higher temperature at which the deprotection enzyme is active. Once deprotection has occurred, the temperature is lowered again to activate the peptide-coupling enzyme and thereby obtain an additional peptide-coupling. This temperature control process continues until the peptide of desired length has been formulated.

In some embodiments of the invention where the first temperature is a lower temperature than the second temperature, the peptide-coupling enzyme is inactivated at the higher (i.e., second) temperature. For example, a deprotection enzyme of the invention may be active at about 50° C. to about 105° C. while a peptide-coupling enzyme of the invention may be active at about 16° C. to 45° C. In this embodiment, an amount of peptide-coupling enzyme sufficient to add another amino acid carboxyl component to the growing peptide is added to the reaction vessel each time the reaction temperature is cooled to the first temperature. An “amount sufficient to add another amino acid carboxyl component” is readily determinable by any ordinarily skilled scientist, and is simply an amount that results in addition of an amino acid carboxyl component to the growing peptide.

In one non-limiting embodiment of the invention, where the first temperature is a lower temperature than the second temperature, the protein coupling enzyme, while being inactive at the second temperature, is not irreversibly inactivated at the second temperature, such that when the temperature of the reaction is changed from the second temperature back to the first temperature, the protein coupling enzyme is once again active. Thus, the invention obviates the need to add new protein coupling enzyme at each cycle (i.e., at each step in which a new amino acid is added to the growing peptide). In some embodiments, the peptide-coupling enzyme is thermally stable at the second (i.e., higher) temperature. For example, the peptide-coupling enzyme that catalyzes peptide-coupling may reversibly folds back into its active position when the temperature is lowered again. Accordingly, once the temperature is lowered, another amino acid carboxyl component is added to the growing peptide without the addition of peptide-coupling enzyme to the reaction vessel.

In some embodiments of the invention, at least one of the peptide-coupling enzyme and the deprotection enzyme is from a thermophilic organism (e.g., a thermophilic prokaryote). By “from a thermophilic organism” is meant that the enzyme (e.g., either deprotection enzyme or peptide-coupling enzyme) has an amino acid sequence identical to the amino acid sequence of the enzyme that naturally occurs in a thermophilic organism. Such an enzyme is obtainable by isolating the naturally-occurring enzyme from the thermophilic organism. Of course, the ordinarily skilled artisan will understand that such an enzyme can also be obtained by standard recombinant molecular biology techniques (e.g., by expressing the nucleic acid encoding such an enzyme is a host organism) (see standard methods, e.g., in Ausubel et al., Current Protocols in Molecular Biology, John Wiley & Sons, New York, NY, 1994, and updates up to and including the year 2001). Such an enzyme can also be synthesized chemically.

In certain embodiments of the invention, the peptide-coupling enzyme or the deprotection enzyme is derived or isolated from a member of the Archaea domain of prokaryotes. For example, a peptide-coupling enzyme or deprotection enzyme of the invention (or a nucleic acid encoding these enzymes) is isolated from a member of the Crenarchaeota kingdom of microbial species, or from a member of the genus Thermoplasma.

The following examples illustrate the preferred modes of making and practicing the present invention, but are not meant to limit the scope of the invention since alternative methods may be utilized to obtain similar results.

### EXAMPLE 1

To identify a peptide-coupling enzyme and a deprotection enzyme according to the present invention, a high throughput assay is developed to discover enzymes which are appropriate for the peptide synthesis methodology of the invention.

Peptide-coupling Enzyme

A serine hydrolase is a suitable template for the desired peptide ligase activity. For a donor of the formula,

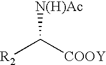

either a methyl ester (Y=CH₃) or ethyl ester (Y=CH₂CH₃) donor is used. Since methoxide is a better leaving group than hydroxide, the rate of formation of the intermediate acyl enzyme is accelerated. The release of methanol upon peptide-coupling helps to drive the reaction to completion.

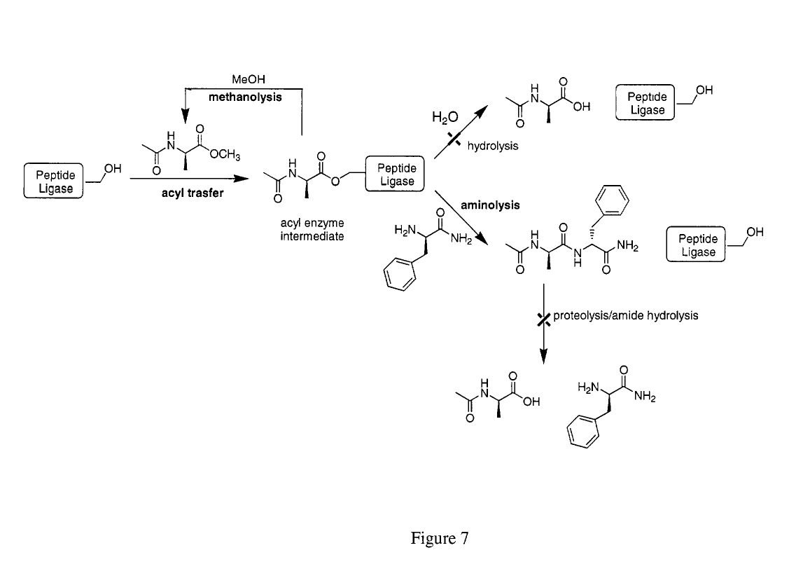

In order to make the ligation efficient, a number of potential side reactions are eliminated. FIG. 7 depicts an acyl-transfer reaction catalyzed by a peptide ligase. Starting with the typical serine hydrolase template for which catalysis proceeds by way of an acyl enzyme intermediate, a number of reactions may follow formation of the active acyl enzyme. Thus, a peptide ligase with a high aminolysis to hydrolysis activity and with high acyl-transferase to proteolytic activity is useful for this approach.

In FIG. 7, the N-acetyl amino acid methyl ester reacts with the enzyme's catalytic serine residue to form an acylenzyme intermediate by way of a tetrahedral transition state. The rate of formation of, and the fate of, the reactive acylenzyme determines the efficiency of the ligation reaction. Nucleophilic attack on the formed acyl enzyme intermediate by water effects the release of (Ac)NH-Ala and effectively destroys the acceptor amino acid electrophile. By contrast nucleophilic attack on the formed acyl enzyme intermediate by the added amino amide Phe-C(O)NH₂ is a productive route which yields the dipeptide product. Thus, an enzyme with the desired peptide ligase activity preferably has a high ratio of aminolysis to hydrolysis activity.

An alternative method to remove background hydrolysis by conducting the reaction in methanol, is developed. Methanolysis of the acylenzyme intermediate regenerates the starting methyl ester. An engineered protease with activity in organic solvent has been described (Economou et al., Biotechnol. Bioeng. 39(6): 658, 1992). Proteolysis of the formed dipeptide constitutes an additional side reaction which is preferably avoided. Thus, a peptide ligase of the invention preferably exhibits both a high ratio of acyltransferase to proteolysis activity and a high ratio of aminolysis to hydrolysis activity, as well as activity and stability in methanol.

A peptide ligase useful in the peptide synthesis method of the present invention has broad substrate scope such that any amino acid sequence can be generated simply by the order of addition of the (Ac)-HN-AA-OMe building blocks. Since the donor amino acid (AA) binds in the S₁ pocket (as shown in FIG. 4), and the nascent acceptor peptide or amino acid binds in the S₁′ to S₂′) pockets (also on FIG. 4), the substrate scope of each of these sites is broad with respect to the side chains of the AA residues accepted. As shown in FIG. 4, the S₂, S₃etc. enzyme subsites are not utilized in this system.

In general, bacterial proteases have broad-substrate scope. In addition to the broad side chain tolerance, it is desirable to incorporate amino acids having the D-configuration into synthetic peptides. The approach of the invention is also applicable to the discovery and/or engineering of one enzyme with P₁ L-configuration enantiospecificity and another with P₁D-configuration enantiospecificity. If both are present in the bioreactor then either configuration amino acid may be incorporated.

Due to the nature of the proposed thermal control of the peptide-coupling and deprotection steps of the invention, the ligase is preferably stable to multiple thermal cycles and retains activity at 50° C. It is not necessary that the ligase be inactive at room temperature. Moreover, performing the ligation reactions in methanol may be useful, in which case an enzyme selected or engineered according to the invention preferably has high activity in organic solvent.

Assay for Peptide-coupling Enzyme:

Since at the macroscopic level it is not possible to differentiate a candidate enzyme with high aminolysis to hydrolysis activity versus one with high acyltransferase to proteolysis activity, in the primary activity screen these properties are selected simultaneously. Gene and enzyme libraries (e.g., those described PCT Publication No. WO 97/04077 and U.S. Pat. No. 5,958,672) are screened with respect to their ability to catalyze, in parallel, the ligation of the four pairs of amino acids (see Table 1). Since it is desirable to discover enzymes that can incorporate either D- or L-amino acids, or alternately one enzyme for each of these applications, the amino acid building blocks used in the initial screen are racemic. While the required peptide ligase is preferably active at 50° C., activity screens are conducted at a range of temperatures from 15° C. to 60° C. (e.g., 15, 25, 35, 45, 60° C.), such that no enzyme with potential ligase activity is overlooked.

TABLE 1
Amino acids that can be used for primary activity screen.

| Amino Acid Donor | Amino Acid Acceptor | Dipeptide Product |
|---|---|---|
| (Ac)HN-(±)-Ala-OMe | H₂N-(±)-Ala-C(O)NH₂ | (Ac)HN-(±)-Ala-Ala-C(O)NH₂ |
| (Ac)HN-(±)-Phe-OMe | H₂N-(±)-Ala-C(O)NH₂ | (Ac)HN-(±)-Phe-Ala-C(O)NH₂ |
| (Ac)HN-(±)-Arg-OMe | H₂N-(±)-Ala-C(O)NH₂ | (Ac)HN-(±)-Arg-Ala-C(O)NH₂ |
| (Ac)HN-(±)-Glu-OMe | H₂N-(±)-Ala-C(O)NH₂ | (Ac)HN-(±)-Glu-Ala-C(O)NH₂ |

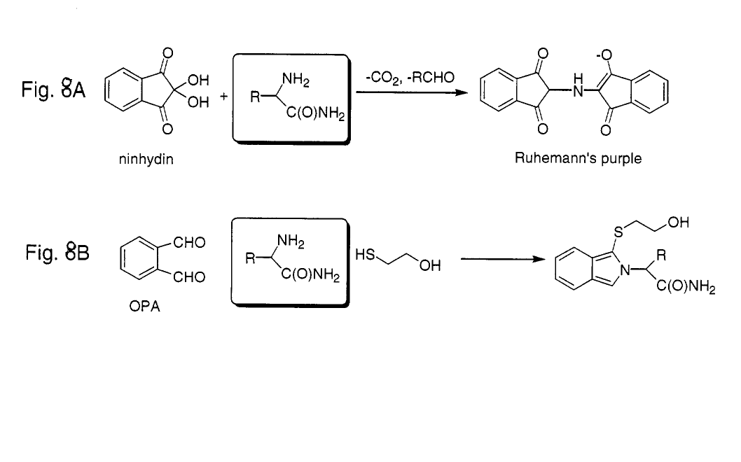

Since successful ligation would effect the loss of a free amine group on the donor amino acid, a free amine based detection system is utilized to assess the extent of peptide ligation. For example, ninhydrin (Bottom et al., Biochem. Educ. 6: 4, 1978) which gives rise to a purple color (570 nm), or alternatively the more sensitive fluorescamine (Udenfriend et al., Science 178: 871, 1972) or ortho-phthalaldehyde (OPA) (Bebson and Hare, Proc. Natn. Acad. Sci. USA 72: 619, 1975) assays which produce highly fluorescent compounds are utilized (see FIGS. 8A and 8B). An often utilized modification of the ninhydrin staining method is the Kaiser test (Kaiser et al., Anal Biochem 34: 595, 1970). In addition, the chloranil test (Christensen, T., Acta Chem. Scand, 1979, 33B, 763) the TNBS test (Hancock and Battersby, Anal. Biochem. 71: 260, 1976) and the picric acid test (Gisin, B. F., Anal. Chim Acta 58: 248, 1972), which are commonly used to monitor coupling in chemical peptide synthesis, are employed. The feasibility of each of these colorimetric and flourometric assays is assessed with respect to amenability to use with a high throughput robotic assay equipment that includes temperature and humidity controlled incubators, liquid handling devices, bar-coding devices, and a variety of plate readers.

In addition, a mass spectrometry (MS)-based assay is used to directly quantify the amount of dipeptide product generated both in a primary discovery screen and also used to asses secondary characterization of the properties of the discovered enzymes. This MS-based assay employs an API 4000 LC-MS system consisting of one API 4000 triple-quad Mass Spectrometer (Applied Biosystems, Forster City, Calif.) in line with a Leap-PAL HTS autosampler (Leap Technologies, Carrbora, N.C.), and two sets of Shimazu 1OA HPLC systems (Shimazu Instrument, Columbia, Md.); one LCQ Advantage LC-MS system consisting of one LCQ Advantage Mass Spectrometer (ThermoFinnigan, San Jose, Calif.) and one Agilent 1100 HPLC system (Agilent Technologies, Willminton, Del.). These instruments are capable of quantitative analysis, and in general each analysis requires less than two minutes per sample. One of the advantages of using LC-MS is the elimination of interference by cellular material present in the screening samples. Since the product and starting materials utilized in the peptide synthesis method of the invention are relatively simple, sensitivity is not expected to be problematic. If necessary, methanol extraction is utilized to remove interfering cellular material and salts at the beginning of the run.

In addition, Multiplex interfaced (MUX) LC-MS system as well as DIOS (Desorption/ionization on Silicon)—TOF techniques are also employed to further enhance screening throughput (Shen et al., Analytical Chemistry 33: 179-187, 2001).

Deprotection Enzyme

In accordance with the invention, a deprotection enzyme (e.g., a deacetylase) can specifically hydrolyze acetyl amides, does not have any proteolytic activity, is inactive at 50° C., is active at 20° C., and/or is thermally tolerant, such that it can undergo reversible inactivation at 50° C. In addition, it preferably has a broad amino acid (peptide) substrate scope. Preferably, the invention features a deacetylase which is active either on both L- and D-acetylated amino acids; alternately, two deacetylases, each of which has activity on either L- and D-acetylated amino acids, are employed in the method of the invention.

Growth selection is one of the most powerful methods for enzyme discovery. In this method, the substrate of choice acts as a nutrient source for the host cells only when those cells contain the enzyme activity of interest, allowing them to grow selectively. Thus, a growth selection is established using a cell strain which works with the environmental genomic libraries. Growth selections are performed in the presence of acetylated peptide (see Table 2) as the sole carbon source.

TABLE 2
Carbon Source Selection for Deacetylases

| | Carbon Sources |
|---|---|
| Selection Substrate | Released by Deacetylase |
| (Ac)NH-(±)-Ala-HN-(±)-Ala-C(O)NH₂ | AcOH, Ala, Ala-C(O)NH₂ |
| (Ac)NH-(±)-Phe-HN-(±)-Ala-C(O)NH₂ | AcOH, Phe, Ala-C(O)NH₂ |
| (Ac)NH-(±)-Arg-HN-(±)-Ala-C(O)NH₂ | AcOH, Arg, Ala-C(O)NH₂ |
| (Ac)NH-(±)-Glu-HN-(±)-Ala-C(O)NH₂ | AcOH, Glu, Ala-C(O)NH₂ |

Growth selections are also performed in the presence of amino acid substrates supplied in M9 salts (Maniatis et al., Molecular Cloning: A Laboratory Manual, 2^{nd} Ed., Cold Spring Harbor Laboratory 1989) as the sole carbon source. This permits a very facile and high throughput discovery for deactylases which are functional at the screening temperature which is set, for example, from 25 or 30° C. (see FIG. 9). In one embodiment, where the desired optimum temperature is 20° C., since bacterial growth may be too slow at this temperature, the selection may conducted at up to about 30° C.

An engineered strain of E. coli (e.g., E. coli strain DH10B (Gibco BRL), which is compatible with the environmental genomic libraries, can grow efficiently on acetate as a sole carbon source. In an alternative approach, an amino acid auxotroph is used to effect genetic complementation. In addition, if background proteolysis be problematic, cell strain engineering is performed.

The discovered deacetylases are subjected to further characterization in an activity-based screen. The deacetylases are characterized with respect to activity on the range of substrates outlined (see Table 2 above) at both 20° C. and at 50° C. The assays are performed in a microtiter plate based format using one of the amine detection systems described above (see FIGS. 8A and 8B). Mass spectral analysis is utilized to further evaluate the activity of the candidate deacetylase enzyme.

Primary Amidase

The final deprotection step to release the free C-terminal carboxylate employs a primary amidase activity which preferably does not possess secondary amidase activity.

### EXAMPLE 2

The efficiency of conducting peptide ligation either in water, methanol cosolvent or pure methanol system is determined. Candidate peptide ligase enzymes, such as esterases or amidases, from commercial sources, publicly available sources, or generated according to the methods of the invention are used to conduct these studies. In addition, a recently reported thermophilic esterase from Bacillus mycoides which has maximal activity at 47° C. (Sugihara et al, J. Biochem. 130(1): 119-126, 2001) is also used to conduct these studies. This enzyme was reportedly able to catalyze the formation of homo-oligomers from D-Phe, D-Trp, D-Tyr and D-Asp methyl esters and exhibited no peptidase activity (Sugihara et al., J. Biochem. 130(1): 119-126, 2001).

For the experiment using the Bacillus mycoides thermophilic esterase as the peptide-coupling enzyme, the Ac-NH-Phe-OMe donor and the Ac-NH-Ala-NH₂ acceptor building blocks are surveyed. A glass or polypropylene vial is used as the reactor. The product is isolated by standard organic extractive methods. Product formation is determined by liquid chromatographic-mass spectral analysis (LC-MS) or nuclear magnetic resonance spectroscopy (NMR), according to standard methods.

Another peptide-coupling enzyme is a muramoyl peptide synthetase. Experiments using muramoyl peptide synthetase employ the D-Ala-D-Ala ligase. In this case, the donor amino acid is effectively activated as its AMP ester which is generated in situ from ATP.

### EXAMPLE 3

The efficiency of conducting deprotection in either in water, methanol cosolvent or pure methanol system is evaluated. Candidate deacetylase enzymes from commercial sources, publicly available sources, or generated according to the methods of the invention are used to conduct these studies. The dipeptides (Ac)HN-(±)-Ala-Ala-C(O)NH₂, (Ac)HN-(±)-Phe-Ala-C(O)NH₂, (Ac)HN-(±)-Arg-Ala-C(O)NH₂, and (Ac)HN-(±)-Glu-Ala-C(O)NH₂ are used to perform these deacetylase studies. Product formation is determined by LC-MS or NMR spectroscopy.

### EXAMPLE 4

In order to form a peptide in three vessels, a protected amino acid carboxyl component and an amino acid nucleophilic component are combined in a first vessel with lipase (a non-limiting peptide-coupling enzyme of the invention). A sufficient amount of lipase is added to the first vessel to allow the lipase to form a peptide bond between the protected amino acid carboxyl component and amino acid nucleophilic components. To obtain lipase activity (to form the peptide bond), the first vessel is incubated at a temperature at which the lipase is active. The temperature may differ depending upon what lipase is used (i.e., from which type of organism the lipase is from), but generally if the lipase is from a non-thermophilic organism (e.g., E. coli bacteria), then the temperature may range from about 16° C. to about 45° C.; whereas, if the lipase is from a thermophilic organism (e.g., Thermus aquaticus), then the temperature may range from about 50° C. to about 105° C.

After the peptide bond is formed (as determined using standard techniques, such as NMR, mass spectroscopy and also the OPA and ninhydrin assays described herein), the resulting protected dipeptide is transferred to a second vessel containing a sufficient amount of carboxypeptidase (a non-limiting deprotection enzyme of the invention) to deprotect the dipeptide. To obtain deprotection activity (to deprotect the dipeptide), the second vessel is incubated at a temperature at which the lipase is active. The temperature may differ depending upon what carboxypeptidase is used (i.e., from which type of organism the carboxypeptidase is from), but generally if the carboxypeptidase is from a non-thermophilic organism (e.g., E. coli bacteria), then the temperature may range from about 16° C. to about 45° C.; whereas, if the carboxypeptidase is from a thermophilic organism (e.g., Thermus aquaticus), then the temperature may range-from about 50° C. to about 105° C.

Once deprotection is accomplished (as determined using standard techniques, such as NMR, mass spectroscopy and also the OPA and ninhydrin assays described herein), the resulting deprotected dipeptide is transferred to a third vessel containing a protected amino acid carboxyl component and a sufficient amount of lipase to allow the lipase to form a peptide bond between the protected amino acid carboxyl component and the deprotected dipeptide. Again, the third vessel is incubated at a temperature at which the lipase is active. After the peptide bond is formed, the resulting protected tripeptide is transferred back to the second vessel containing carboxypeptidase to deprotect the tripeptide. The deprotected tripeptide is then transferred back to the second vessel containing lipase to add a protected amino acid carboxyl component to obtain a four amino acid long protected peptide. The peptide is transferred back and forth between the second and third vessels to obtain a peptide of desired length. Note that if a protected peptide is desired, the final vessel used is the third vessel (containing lipase). If a deprotected peptide is desired, the final vessel used is the second vessel (containing carboxypeptidase).

### EXAMPLE 5

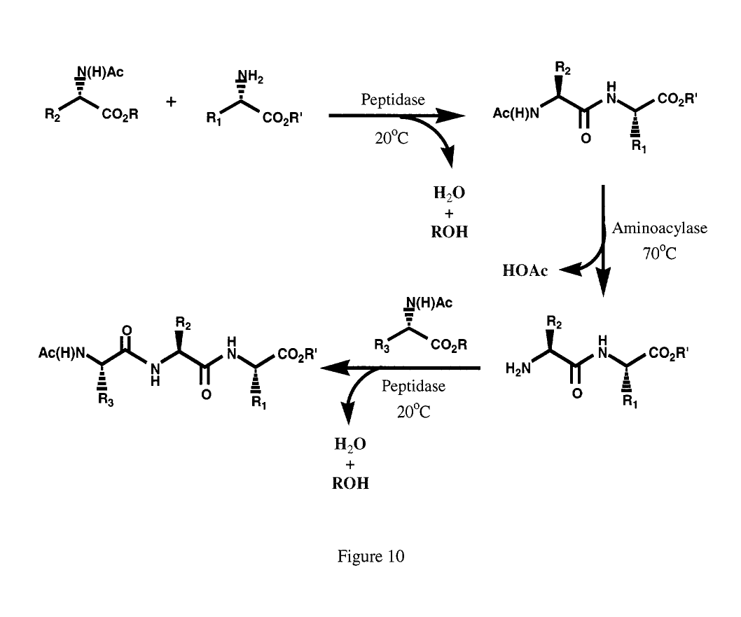

In order to form a peptide in a single vessel, using an enzymatic process, two amino acids (e.g., about 10 mM to 5 M of each) are combined in a single vessel with two enzymes, peptidase (a non-limiting peptide-coupling enzyme of the invention) and aminoacylase (a non-limiting deprotection enzyme of the invention). One amino acid is protected with a protecting group, which is catalyzed (i.e., deprotected) by the aminoacylase at 70° C. The temperature of the reaction vessel is brought to 20° C., and the coupling of the amino acids is catalyzed by the peptidase. The waste products of the reaction are water and ROH (see FIG. 10). The temperature of the reaction vessel is then raised to 70° C., and the dipeptide deprotection is catalyzed by the aminoacylase. The temperature is then lowered again to 20° C., where another amino acid is added to the dipeptide, and is catalyzed by peptidase to produce a tripeptide (see FIG. 10).

Note that in FIG. 10, the catalytic temperatures of the enzymes can be reversed. Thus, the aminoacylase can be active at 70° C. while the peptidase can be active at 20° C., with thermocying used to control synthesis of the growing peptide.

### EXAMPLE 6

The active form of neurotensin is a thirteen amino acid long peptide having the sequence ELYENKPRRPYWL (SEQ ID NO: 1) (see, e.g., Hammer et al., J. Biol. Chem. 255(6): 2476-2480, 1980; Williamson et al., Protein Expression and Purification 19: 271-275, 2000). This thirteen amino acid long peptide is made according to the methods of the invention using solid-phase peptide synthesis. To do this, the C-terminal amino acid, here leucine, attached to a solid-phase substrate using standard methods (Merrifield, R. B., Science 150(3693):178-185, 1965 and Merrifield, R. B., Anal Chem 38(13): 1905-1914, 1966). The solid-phase substrate attached leucine residue is the “amino acid nucleophilic component” in accordance with the invention (see, e.g., FIG. 1). This solid-phase substrate attached leucine residue is inserted into a vessel (note that the solid-phase substrate may be the wall of the vessel itself, and the leucine residue is simply attached to the wall of the vessel), and is combined with a protected isoleucine residue, which is the “amino acid carboxyl component” of the invention, and a sufficient amount of a peptide-coupling enzyme (e.g., peptidase) to form a peptide bond between the leucine residue and the protected isoleucine residue. Note that because the isoleucine residue is protected, the peptide-coupling enzyme will not form a peptide bond between two isoleucine residues. The product of the first reaction is the IL dipeptide, where the isoleucine residue is protected and the leucine residue is attached to the solid-phase substrate.

To add the next N-terminal amino acid of neurotensin, tyrosine, the isoleucine residue is first deprotected. Where the solid-phase support to which the dipeptide is attached is the vessel itself, the vessel is rinsed to remove the peptide-coupling enzyme and any remaining unincorporated protected isoleucine residues. Alternatively, where the solid-phase support to which the dipeptide is attached is removable from the vessel, the support is simply lifted out of the vessel and rinsed to remove the peptide-coupling enzyme and any remaining unincorporated protected isoleucine residues.

Next, the protected dipeptide is combined with a deprotection enzyme (e.g., aminoacylase) to deprotect the isoleucine residue of the dipeptide. The deprotected dipeptide is then rinsed to remove the deprotection enzyme, and then combined with the peptide-coupling enzyme and protected tyrosine residues. The resulting YIL tripeptide is deprotected, and the next amino acid residue, proline, is added.

In this manner, the neurotensin peptide is synthesized, C-terminally to N-terminally. The final amino acid added to the growing peptide, glutamic acid, is the N-terminal amino acid of the neurotensin peptide. If it is desired that the resulting neurotensin peptide be protected, the final deprotection step is simply not performed.

The final product, which is either protected or deprotected neurotensin peptide, is now ready to be removed from the substrate according to standard methods and used as desired (e.g., administered to a patient suffering from schizophrenia or Parkinson's disease (see, e.g., Williamson et al., Protein Expression and Purification 19: 271-275, 2000).

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A method for synthesizing a peptide, comprising:
(a) ligating a protected amino acid carboxyl component to an amino acid nucleophilic component with a peptide-coupling enzyme active at a first temperature to produce a first peptide;
(b) deprotecting the first peptide with a deprotection enzyme active at a second temperature; and
(c) ligating a protected amino acid carboxyl component to the deprotected first peptide with the peptide-coupling enzyme active at the first temperature to produce a second peptide.

2. The method of claim 1, wherein steps (b) and (c) are repeated to obtain a peptide of a desired length.

3. The method of claim 1, wherein the protected amino acid carboxyl component is a N-acyl protected amino acid.

4. The method of claim 3, wherein the acyl is selected from the group consisting of acetyl, formyl, benzoyl, and carbamoyl.

5. The method of claim 1, wherein the protected amino acid carboxyl component consists of the formula,

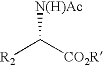

6. The method of claim 5, wherein the R′ group of the protected amno acid carboxyl component is selected from the group consisting of hydrogen and alkyl.

7. The method of claim 1, wherein the amino acid nucleophilic component is a C-terminal protected amino acid.

8. The method of claim 1, wherein the amino acid nucleophilic component consists of the formula,

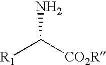

9. The method of claim 8, wherein the R″ group of the amino acid nucleophilic component is t-butyl.

10. The method of claim 1, wherein the peptide-coupling enzyme does not have protease activity.

11. The method of claim 1, wherein the peptide-coupling enzyme is selected from the group consisting of a peptidase, a ligase, a lipase, a protease, a carboxypeptidase, and an amidase.

12. The method of claim 1, wherein the deprotection enzyme is selected from the group consisting of a deacetylase, an aminoacylase, an amidase, a carbamate and a carboxypeptidase.

13. The method of claim 2, wherein the desired length is from about two to about forty amino acid residues in length.

14. The method of claim 13, wherein the desired length is from about three to about thirty amino acid residues in length.

15. The method of claim 1, wherein the method is performed in a single reaction vessel.

16. The method of claim 1, wherein the peptide-coupling enzyme and the deprotection enzyme are not active simultaneously.

17. The method of claim 16, wherein the first temperature is higher than the second temperature.

18. The method of claim 16, wherein the first temperature is lower than the second temperature.

19. The method of claim 16, wherein the peptide-coupling enzyme is not irreversibly inactivated at the second temperature.

20. The method of claim 16, wherein the deprotection enzyme is not-irreversibly inactivated at the first temperature.

21. The method of claim 1, wherein the peptide-coupling enzyme is derived from a thermophilic organism.

22. The method of claim 1, wherein the deprotection enzyme is derived from a thermophilic organism.

23. The method of claim 1, wherein the deprotection enzyme is inactive at the first temperature.

24. The method of claim 1, wherein the peptide-coupling enzyme is inactive at the second temperature.

25. A method for identifying a deprotection enzyme comprising culturing a plurality of host cells on a substrate comprising an acetylated peptide as a sole carbon source, wherein each host cell of the plurality comprises a nucleic acid molecule suspected of encoding a deprotection enzyme, and wherein a host cell that grows on the substrate is identified as a host cell that expresses a deprotection enzyme.
